(19) United States
(12) Patent Application Publication
(10) Pub. No.: US 2024/0369582 A1
(43) Pub. Date: Nov. 7, 2024

(54) CLOTTING TIME COMPUTATION METHODS AND SYSTEMS INCLUDING ATTACHMENT DEVICES FOR SMARTPHONES

(71) Applicant: University of Washington, Seattle, WA (US)

(72) Inventors: Justin Chan, Seattle, WA (US); Shyamnath Gollakota, Seattle, WA (US); Kelly Michaelsen, Seattle, WA (US)

(73) Assignee: University of Washington, Seattle, WA (US)

(21) Appl. No.: 18/557,905

(22) PCT Filed: May 20, 2022
(86) PCT No.: PCT/US2022/030326

§ 371 Date: Oct. 27, 2023

Related U.S. Application Data
(60) Provisional application No. 63/191,598, filed on May 21, 2021.

(51) Int. Cl. G01N 33/86; B01L 3/00

(52) U.S. Cl. CPC G01N 33/86; B01L 3/508; B01L 2200/16; B01L 2300/0654; B01L 2400/0433; G01N 2333/7454

(57) ABSTRACT

Example systems may include an electronic device (e.g., a smartphone) and an attachment member that may couple the electronic device to a cup. The cup may be positioned within a field of view (e.g., within a focal length) of a camera that is coupled to or integral to the electronic device. The cup may receive a sample of whole blood and/or plasma. The attachment member may convey vibration from the electronic device to the cup. The cup may include a particle positioned in the cup. The camera may capture images of the cup including the particle, and motion of the particle may be used by the electronic device to calculate a clotting time of the sample. For example, the particle may move freely responsive to vibration during a time the sample is in a non-clotted state, but may slow and/or stop motion when the sample has clotted.

### CROSS-REFERENCE TO RELATED APPLICATIONS

This application claims the benefit under 35 U.S.C. § 119 of the earlier filing date of U.S. Provisional Application Ser. No. 63/191,598 filed May 21, 2021 the entire contents of which is hereby incorporated by reference in its entirety for any purpose.

### TECHNICAL FIELD

Examples described herein relate to measuring clotting time of a sample using motion of a particle in the sample during vibration. Examples of attachment members are described that may position a sample cup within a field of view of a smartphone camera and transmit vibration from the camera to the sample cup.

### BACKGROUND

The human body responds to injury with bleeding, followed by clot formation and eventually lysis. This carefully maintained homeostasis minimizes the risks of hemorrhage and inappropriate clotting like ischemic stroke, myocardial infarction or pulmonary embolus. However, for millions of people, medical conditions such as atrial fibrillation, mechanical heart valves and genetic mutations increase the risk of morbidity and mortality from blood clotting. These individuals may require lifelong administration of anticoagulation drugs such as warfarin, an effective medication but also one of the most common causes of hospitalization due to adverse drug events. Hence, medication effects must be closely monitored via frequent monitoring of coagulation properties (e.g., clotting time), such as prothrombin time (PT) or international normalized ratio (INR) tests to assess coagulation properties due to the drug's narrow therapeutic index and interactions with food and other medications. While newer anticoagulants that do not rely on regular PT/INR testing are increasing in popularity, studies show that warfarin remains the most commonly prescribed outpatient blood thinner.

PT/INR testing monitors extrinsic and common pathways of the coagulation cascade. These tests are usually performed in a laboratory on expensive equipment after separating plasma from whole blood. Home PT/INR monitors directly utilize blood and have been shown to lengthen the amount of time spent in the therapeutic range, decrease the risk of thromboembolism in young patients and improve patient satisfaction and quality of life. Time spent in the therapeutic window benefits patients with non-valvular atrial fibrillation on warfarin, since the risk of bleeding is five times higher with overly aggressive anticoagulation and the risk of ischemic events is three times higher with insufficient anticoagulation compared to levels in the therapeutic range.

Despite the existence of several home PT/INR testing modules, access to affordable and accurate PT/INR testing remains a challenge. Patients in the United States are in the therapeutic range only about 64% of the time. Patients in developing countries like Botswana, Uganda, and India are in this range, only 40% of the time due to less frequent testing. Despite the potential for improvements from home PT/INR testing9, these devices cost hundreds of dollars, limiting their utility in resource-constrained environments.

### SUMMARY

Examples of smartphone attachments are described herein. An example smartphone attachment may include a cup and an attachment member configured to contact a bottom and a side of the cup and a smartphone. The attachment member may be configured to transfer vibration from the smartphone to the cup. The attachment member may be further configured to position the cup within a field of view of a camera of the smartphone. The example smartphone attachment may further include a particle positioned in the cup and configured to move within the cup responsive to vibration from the smartphone.

In some examples, the particle may be a copper wire. In some examples, the particle may be an iron particle.

In some examples, the cup is configured to receive a sample. In some examples, a motion of the particle slows responsive to clotting of the sample.

Examples of methods are described herein. An example method may include vibrate, using a motor of a smartphone, a container enclosing a sample and a measurement particle. The method may also capture, using a camera of the smartphone, images of the measurement particle during vibration. The method may also measure motion of the particle based on the images, and calculate a clotting time based on the motion.

In some examples, the measurement particle may be a copper particle or an iron particle.

In some examples, calculate the clotting time includes determining a prothrombin time (PT) or international normalized ratio (INR).

In some examples, the sample includes a whole blood sample or a plasma sample.

In some examples, the clotting time is calculated based on when the motion of the particle stops.

In some examples, the motion slows responsive to clotting in the sample.

In some examples, the motion is measured during a non-clotted state of the sample.

Examples of systems are described herein. An example system may include a mobile terminal comprising a processor, a vibration motor, and a camera. The example system may also include an attachment member configured to couple the mobile terminal to a cup. The example system may also include a particle positioned in the cup and configured to move within the cup responsive to vibration from the mobile terminal. The vibration motor may be configured to vibrate the attachment member. The camera may be configured to capture images of a sample and the particle in the cup. The processor may be configured to calculate a clotting time of the sample in the cup based on the images.

In some examples, the mobile terminal includes a smartphone.

In some examples, the attachment member is further configured to position the cup within a field of view of the camera.

In some examples, the particle includes a copper particle. In some examples, the particle includes an iron particle.

In some examples, the processor is configured to calculate the clotting time based on a time when motion of the particle stops.

In some examples, the processor is configured to calculate the clotting time based on a slowing of a motion of the particle.

In some examples, the sample includes a whole blood sample or a plasma sample.

### BRIEF DESCRIPTION OF THE DRAWINGS

FIG. 1 is a schematic illustration of a system arranged in accordance with examples described herein.

FIG. 2 is an image of an implemented example system arranged in accordance with examples described herein, together with a top-down view of the sample cup.

FIG. 3 includes a sequence of images demonstrating image processing techniques used to isolate a cup holder and particle movement in accordance with example systems described herein.

FIG. 4 is a plot of particle amplitude over time during operation of an example system arranged in accordance with examples described herein.

### DETAILED DESCRIPTION

Certain details are set forth herein to provide an understanding of described embodiments of technology. However, other examples may be practiced without various of these particular details. In some instances, well-known circuits, control signals, timing protocols, and/or software operations have not been shown in detail in order to avoid unnecessarily obscuring the described embodiments. Other embodiments may be utilized, and other changes may be made, without departing from the spirit or scope of the subject matter presented here.

Tests which wholly or partially utilize a measurement of clotting time, such as prothrombin time (PT) and/or international normalized ratio (INR) testing, may be critical for millions of people on lifelong anticoagulation with warfarin. Currently, testing is performed in hospital laboratories or with expensive point-of-care devices limiting the ability to test frequently and affordably. Since manual visual detection of clot time is subjective, requires training and can vary between operators and institutions, existing tests use optical and magnetic Hall-effect sensors to automatically track changes in plasma viscosity and turbidity. Mechanical approaches use specialized hardware to analyze the movements of steel balls, iron fillings, and magnets in the plasma sample. Other automated approaches include electro-chemical sensors that measure capacitance and resistance, quartz crystal resonators, micro-resonators, and centrifugal-type microfluidic platforms. Though laboratory clotting assays enable high-throughput testing, they may not be able to provide rapid turnaround for anticoagulant therapy in emergency room and intensive care units, which often require results within 30 minutes and around-the-clock availability. Commercial point-of-care systems eliminate these delays but can be expensive for field use, in resource-constrained settings and for in-home patients, who cite cost of PT/INR home devices as the primary barrier to self-testing.

Examples described herein provide systems and methods that may measure a clotting time that uses the vibration motor and camera on smartphones to track micro-mechanical movements of a particle. The clotting time measurement may be used to compute PT/INR. An implemented example system computed the PT/INR with inter-class correlation coefficients of 0.963 and 0.966, compared to a clinical-grade coagulation analyzer for 140 plasma samples and demonstrated similar results for 80 whole blood samples using a single drop of blood (10 μl). When tested with 79 blood samples with coagulopathic conditions, an implemented example smartphone system demonstrated a correlation of 0.974 for both PT/INR. Given the ubiquity of smartphones in the global setting, examples of systems described herein may provide affordable and effective PT and INR testing in low-resource environments.

Systems described herein may use the vibration motor and camera on existing smartphones to perform PT/INR testing. Smartphones are increasingly becoming ubiquitous in resource-constrained environments and developing countries both in rural and urban settings. Vibration motors and cameras have been an integral part of smartphones for more than a decade. Repurposing these smartphone sensors for PT/INR testing could allow for a more affordable blood clot testing tool. Examples of systems described herein may visually track micro-mechanical movements of a particle in a cup with a sample (e.g., either a single drop of whole blood or plasma) and the addition of activators. No additional electronic components are required in some examples beyond a lightweight plastic attachment that couples the phone's vibrations to the cup. When the mixture is in a fluid state, the particle moves freely with the phone's vibration. As the blood clots, the viscous mixture causes the particle to slow to a stationary state. Using computationally efficient video analytic algorithms that run on the smartphone in under 37 ms, in some examples, the particle's motion is analyzed (e.g., in under one minute in some examples) to determine the PT/INR values. Example systems can run on older smartphones such as second-hand iPhone 5s phones that were released in 2013 and cost $35. Making coagulation testing accessible in this manner may help improve time within the therapeutic range for anti-coagulation users, particularly in rural locations.

Examples described herein may leverage on-board vibration motors and cameras that are ubiquitous on modern smartphones and are accessible in resource-constrained environments. This may provide an automated solution that does not require manual observation or interpretation of clotting data.

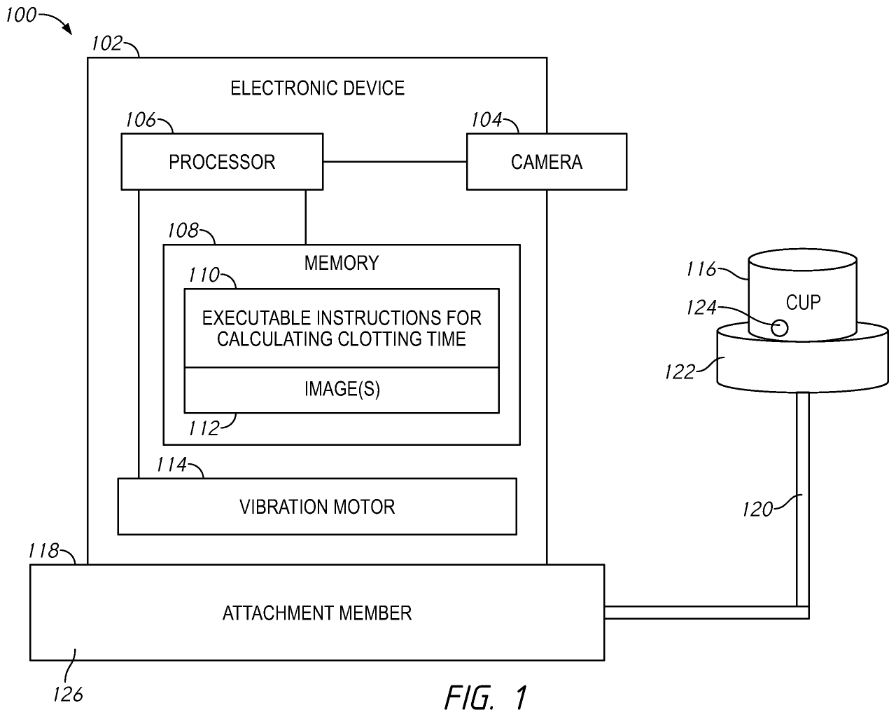

FIG. 1 is a schematic illustration of a system arranged in accordance with examples described herein. The system 100 includes an electronic device 102, an attachment member 118, and a cup 116. The electronic device 102 includes camera 104, processor 106, memory 108, and vibration motor 114. The memory 108 includes executable instructions for calculating clotting time 110 and image(s) 112. The electronic device 102 is coupled to attachment member 118. The attachment member 118 includes device attachment portion 126, arm 120 and cup attachment portion 122. The cup attachment portion 122 is coupled to cup 116. The cup 116 holds particle 124. The components shown in FIG. 1 are shown by way of example. Additional, fewer, and/or different components may be used in other examples.

Examples of systems described herein may accordingly include an electronic device. Electronic devices described herein may generally include one or more sensors (e.g., cameras) and processing capability to perform image acquisition, analysis, and/or calculations described herein. Any of a variety of electronic devices may be used including, but not limited to, one or more computers, servers, or appliances. In some examples, an electronic device may be a mobile terminal (e.g., able to be worn, held, carried, or otherwise transported during normal operation). Examples of mobile terminals may include, but are not limited to, one or more smartphones, tablets, watches, or wearable devices.

Examples of electronic devices described herein may include one or more cameras, such as camera 104 of FIG. 1. The camera may be integral to the electronic device 102 and/or may be electronically coupled to the electronic device 102. The camera 104 may be implemented, for example, using a camera of a smartphone. The camera 104 may be used to capture one or more images. The images in some example may be captured as one or more videos (e.g., each image may be a frame of a video). Accordingly, the camera 104 may capture one or more videos.

In some examples, video frames captured periodically may be used to calculating clotting time or other values described herein. In some examples, frames captured every 100 ms may b used. Other frequencies may be used in other examples (e.g., frames captured every 50 ms, every 75 ms, every 200 ms, or other frequencies). Generally, the camera 104 may be implemented using a camera that can capture video at 10 fps or greater. Modern smartphones are generally capable of capturing video records at a minimum of 24 or 30 fps, which is the standard frame rate for video recordings. A common resolution for video recording on modern smartphones is 1920×1080, which may be used to perform methods described herein.

Examples of electronic devices described herein may include one or more processors, such as processor 106 of FIG. 1. Processors, such as processor 106, may be implemented using one or more central processing units (CPUs), graphics processing units (GPUs), field programmable gate arrays (FPGAs), application specific integrated circuits (ASICs), controllers, microcontrollers, or other circuitry. Processors described herein may be used to implement software and/or firmware systems. For example, the processor 106 may be used to execute one or more executable instructions encoded on computer readable media (e.g., memory). While a single processor 106 is shown in FIG. 1, it is to be understood that any number of processors may be used, and multiple processors may be in communication with each other to perform functions of a processor described herein. The processor 106 may be coupled to (e.g., in communication with), camera 104, memory 108, and/or vibration motor 114 of FIG. 1.

Examples of electronic devices described herein may include memory, such as memory 108 of FIG. 1. Generally, any number or kind of memory may be used including read-only memory (ROM), random access memory (RAM), flash memory, one or more solid state drives, one or more SD cards, or other computer readable media. The memory may be encoded with executable instructions, such as executable instructions for calculating clotting time 110. Memory described herein, such as memory 108 may store data, such as image(s) 112 of FIG. 1. While a single memory 108 is shown in FIG. 1, it is to be understood that any number of memory devices may be used, and the executable instructions and/or data may be distributed across multiple memories accessible to the processor 106.

Examples of electronic devices described herein may include a motor, such as vibration motor 114 of FIG. 1. The motor may cause all or portions of the electronic device 102 to vibrate. For example, the electronic device 102 may have a housing. The housing may enclose processor 106, memory 108, camera 104, and/or vibration motor 114. The housing may be implemented using a plastic, metal, glass, and/or ceramic material. The housing may be vibrated responsive to actuation of the vibration motor 114. Accordingly, the vibration motor 114 may cause the electronic device 102 to vibrate.

Electronic devices described herein, including electronic device 102 may include additional components. For example, the electronic device 102 may include or be coupled to one or more display(s), input device(s) (e.g., keyboards, mice, touchscreens, microphones), speakers, and/or communication interface(s) (e.g., cellular antenna, Wi-Fi, network interface, Bluetooth interface). The additional components may be in communication with processor 106 and/or memory 108 of FIG. 1.

Examples of systems described herein may provide an attachment member that may physically couple to an electronic device. For example, the attachment member 118 of FIG. 1 may couple to the electronic device 102. Generally, the attachment member 118 may be sized to contact portions of a housing of the electronic device 102. For example, the attachment member 118 may surround a portion of a housing of the electronic device 102. A portion of the electronic device 102 may, for example, be inserted into the attachment member 118.

Examples of systems described herein may include a container, such as cup 116 of FIG. 1. The cup 116 may be sized to hold a sample. The sample may include a whole blood sample and/or a plasma sample. The cup 116 is positioned to be within a field of view of a camera, e.g., camera 104 of FIG. 1. In the schematic illustration of FIG. 1, it appears that the camera 104 is viewing a side of the cup 116. In some examples, the camera 104 is positioned to have a top-down view of the cup 116. In some examples, the container may be made of a flexible material. For example, the cup 116 may be formed of silicone. Generally, the container, such as cup 116, may contain any volume of fluid. In some example, the cup may hold up to 50 μl of fluid. In some examples, the cup may hold up to 40 μl of fluid. In some examples, the cup may hold up to 30 μl of fluid. Other volumes may be used in other examples. In some examples, the cup 116 may be disposable (e.g., it may be used for a single sample and replaced with a new cup for a next sample).

Examples of containers described herein, such as cup 116 of FIG. 1 may include an activator substance. For example, a dried activator substance may be disposed in the cup, such as dried thromboplastin. Generally, the activator substance may be a substance which may activate one or more pathways of the coagulation cascade. By containing a dried activator substance, in some examples, an activator substance may not need to be added to the cup during operation. In other examples, however, activator may be added to the container during operation.

Examples of cups described herein, such as cup 116 of FIG. 1, may include one or more particles, such as particle 124 of FIG. 1. In some examples, the particle may be secured to the cup 116. For example, the particle 124 may be secured to the cup 116 for transport or sale of the attachment member 118 and cup 116. However, the particle 124 may be free to move responsive to vibration of the electronic device 102. In some examples, the particle 124 may be disconnected from any cup 116 attachment provided for the purposes of transport before use. In some examples, the particle 124 may be coupled to the cup 116 using a wire or other connector which allows for movement of the particle 124 responsive to vibration of the electronic device 102. Particles described herein may be formed of iron and/or copper. In some examples, a particle may be made of foam. Other materials may be used in other examples.

Particles described herein may generally be of any size. Example particles may be spherical. Other shapes may be used in other examples. In some examples, the particle 124 may be implemented using a wire (e.g., a copper and/or iron wire). Generally, a non-porous material may be used to implement particle 124, which may reduce and/or eliminate absorption of reagent by the particle. In some examples, the particle 124 may be a 1 mm long AWG22 copper wire. Other lengths or thicknesses may be used in other examples. Generally, the particle may be lightweight enough to move freely in the cup responsive to vibration of the electronic device. In some examples, the particle may include coloring(s) or marking(s) to increase visibility of the particle as viewed by the camera 104. For example, the particle 124 may be coated with ink and/or patterned to facilitate recognition of the particle in images captured by the camera. In some examples, the particle 124 may be coated with dark blue ink for increased visibility. Other colors may be used in other examples.

Examples of attachment members described herein may be provided to position a cup within a field of view of a camera and/or to convey (e.g., transfer) vibration from an electronic device to the cup. For example, the attachment member 118 of FIG. 1 includes device attachment portion 126, arm 120, and cup attachment portion 122. The device attachment portion 126 is provided to fit to a portion of a housing of the electronic device 102. The arm 120 is coupled to the cup 116 and is sized to position the cup 116 within a field of view of the camera 104. In the example of FIG. 1, the attachment member 118 has an ‘L’ shaped structure which may be thin to allow the vibrations from the electronic device to propagate to the cup while reducing dampening. The cup attachment portion 122 is sized to accommodate the cup 116 (e.g., fit to the cup 116). The cup attachment portion 122 accordingly may contact a bottom and/or side(s) of the cup 116 to convey vibration from the electronic device 102 to the cup 116. In some examples, by contacting the bottom and/or sides of the cup an improved physical energy transfer may occur from the attachment member 118 to the cup 116. While an ‘L’ shaped structure is shown in FIG. 1, other shapes, sizes, and configurations of attachment members may be used. The particle 124 may move within the cup 116 responsive to vibration of the electronic device 102, which vibration may be transferred (e.g., transmitted) to the cup 116 through the attachment member 118.

Accordingly, a width of attachment member 118 (e.g., a width of the device attachment portion 126 of attachment member 118) may be determined by the dimensions of a housing of the electronic device 102. The width of the device attachment portion 126 may be sized to fit around a width of the electronic device 102. In some examples, the attachment member 118 may be formed using 3D printing techniques. Other formation techniques may be used (e.g., injection molding).

During operation, a sample may be positioned in the cup 116. For example, a whole blood and/or plasma sample may be collected from a patient and inserted into the cup 116. In some examples, a capillary blood sample may be used. The sample may be collected, for example, by lancing a finger or other portion of a patient, drawing blood into a capillary tube or other delivery device, and delivering the sample to the cup. In some examples, the sample may be collected and stored prior to introducing the sample to the cup. In some examples, the sample may be stored for hours (e.g., twelve hours in some examples). In some examples, such as when the sample has been stored prior to delivery to the cup, the sample may be heated prior to introduction to the cup. In some examples, the cup may hold 10-20 μl of sample (e.g., plasma and/or whole blood). Any of a variety of heaters may be used, and in some examples, the heater may be integrated into the electronic device 102 and/or attachment member 118 and/or cup 116. For example, a Peltier device, temperature sensor, transistor, and/or microcontroller may be used to heat the sample. In some examples, the heater may be powered by the electronic device 102 (e.g., using a USB connection to the electronic device 102).

In some examples, an activator substance (e.g., tissue factor) may be disposed in the cup and accordingly may mix with the sample. In some examples, an activator substance may be added to the cup. An example activator substance is thromboplastin, which may activate one or more pathways of the coagulation cascade. In some examples, 10 μl of whole blood may be used and 20 μl of tissue factor may be added.

The electronic device 102 may be vibrated. For example, the vibration motor 114 may be used to vibrate the electronic device 102. In some examples, an application executing on the electronic device 102 may be used to initiate and/or otherwise control vibration of the electronic device 102. Vibration of the electronic device 102 may accordingly cause vibration of the cup 116 containing a sample and a particle 124, as the vibration may be transferred through the attachment member 118. In some examples, the executable instructions for calculating clotting time 110 include instructions for initiating vibration of the electronic device 102 and/or vibrating the electronic device 102 for a predetermined length of time, using vibration motor 114. Accordingly, a container (e.g., cup 116) enclosing a sample and a measurement particle (e.g., particle 124) may be vibrated. When the blood or plasma sample is not coagulated, the electronic device's vibrations cause the particle to move and/or rotate within the sample. Vibration strength may vary in some examples from 1.05 g to 3.77 g in some examples (e.g., as measured using an accelerometer at the cup).

One or more images of the measurement particle may be captured during vibration of the container. For example, the camera 104 may capture one or more images of the particle 124 during vibration of the cup 116. In some examples, the camera 104 may take a video of the particle 124 during vibration. The executable instructions for calculating clotting time 110 may include instructions for capturing images using the camera 104 during a time the vibration is occurring. For example, the camera 104 may begin capturing images when the vibration motor 114 begins vibrating in some examples. In some examples, a user may initiate a start and/or stop of the vibration motor 114 and/or a start and/or stop of image capture using the camera 104. In some examples, the cup may be illuminated during a time that the images are being captured. For example, a flash of the electronic device 102 may be used to illuminate the cup 116.

The electronic device 102 may measure a motion of the particle 124 during vibration of the cup 116. For example, executable instructions for calculating clotting time 110 may include executable instructions for measuring movement of a particle using one or more images captured during vibration. In some examples, motion of the particle may be measured by measuring a distance the particle has traveled between images (e.g., between frames of a video). In some examples, the particle may have a particular velocity during a time that the sample is in a non-clotted state. As the sample clots, however, the motion of the particle may slow. For example, as the sample coagulates, its increasing viscosity may constrict the particle's movements, slowing its 2D motion as seen by the camera 104.

In some examples, a clotting time of the sample may be calculated based on a motion of the particle. For example, a clotting time may be determined as an elapsed time until the movement of the particle stops. In some examples, a clotting time may be determined based on a rate of slowing of the motion of the particle. The executable instructions for calculating clotting time 110 may include instructions for calculating the clotting time based on motion of the particle. In some examples, the executable instructions for calculating clotting time 110 may include instructions for making a PT/INR measurement—for example, calculating PT and INR based on the particle motion. Generally, the particle's movement may be recorded optically by the smartphone camera and analyzed to calculate PT/INR. Other values related to clotting time may be calculated in other examples, such as thromboplastin time (TPP) and/or thrombin time (TT).

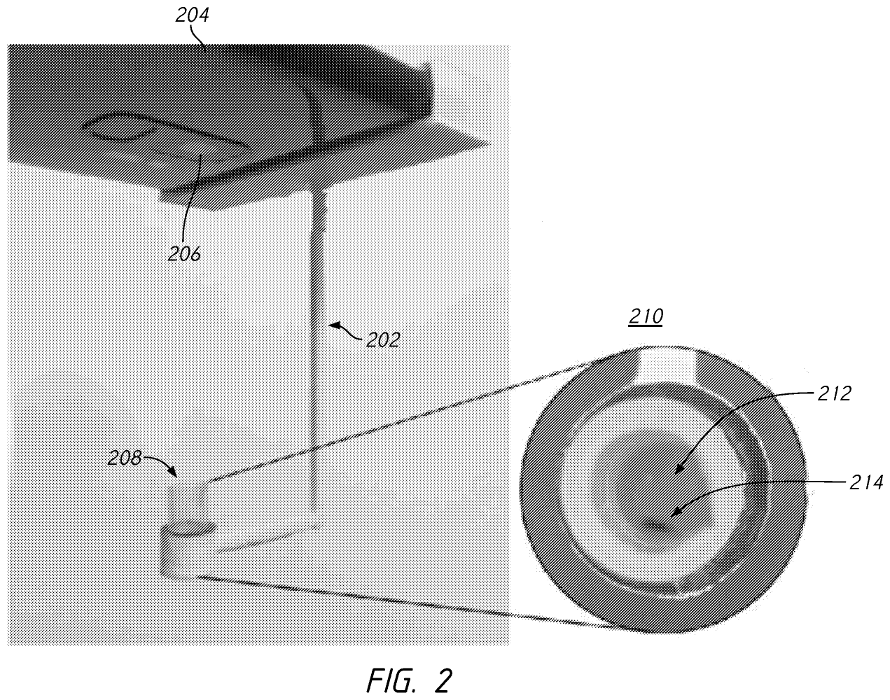

FIG. 2 is an image of an implemented example system arranged in accordance with examples described herein, together with a top-down view of the sample cup. The example of FIG. 2 includes smartphone 204. The smartphone 204 includes camera 206. The smartphone 204 may be fitted into attachment member 202. The attachment member 202 is coupled to cup 208. FIG. 2 also shows top-down view 210 of cup 208. The top-down view 210 of cup 208 may be as viewed, for example, by camera 206. The cup 208 may hold sample 212 and particle 214.

The components shown in FIG. 2 are exemplary only; additional, fewer, and/or different components may be provided in other examples.

The example of FIG. 2 may be implemented using and/or used to implement the system 100 of FIG. 1. For example, the smartphone 204 may implement the electronic device 102 of FIG. 1. Accordingly, the smartphone 204 may include executable instructions for calculating clotting time 110 and other components shown and/or described with reference to electronic device 102. The attachment member 202 may be used to implement and/or may be implemented by the attachment member 118 of FIG. 1. The cup 208 may be used to implement and/or may be implemented by the cup 116 of FIG. 1.

In one example, the particle 214 is a 1 mm long AWG22 copper wire coated with dark blue ink for increased visibility.

Generally, video and/or image processing techniques described herein may include three general actions. Each and/or all action may be performed by one or more processors in accordance with executable instructions (e.g., may be implemented in software). For example the electronic device 102 may perform any and/or all of these actions by executing executable instructions for calculating clotting time 110. The video and/or images analyzed may be captured by camera 104 of FIG. 1. While in the example of FIG. 1, the same electronic device 102 analyzes the images as captures the images, it is to be understood that in other example the images may be captured, stored, and transmitted and/or accessed by another computing system which may perform the analysis actions described herein.

As a first action, electronic devices described herein may identify pixels in a given image (e.g., frame) correspond to the cup holder and/or cup (e.g., cup attachment portion 122 and/or cup 116 of FIG. 1). Doing this provides a determination of which pixels correspond to the interior of the cup where the particle vibrates and which pixels correspond to the cup and cup holder, where the pipetteor capillary tube entering and exiting the frame can be seen in the image(s) (e.g., frame(s)).

As a next action, electronic devices described herein may identify a start of a clotting time measurement (e.g., PT measurement), tₛₜₐᵣₜ, by tracking the motion of the tube or other delivery device as it enters and exits the frame.

As a next action, electronic devices described herein may compute the end of the clotting time measurement (e.g., PT measurement), tend, by tracking the particle inside the cup.

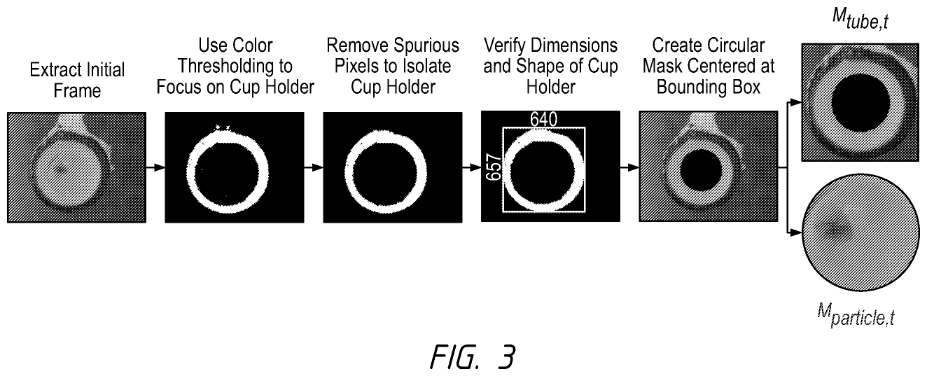

FIG. 3 includes a sequence of images demonstrating image processing techniques used to isolate a cup holder and particle movement in accordance with example systems described herein. The images shown in FIG. 3 may be captured, for example by camera 104 of FIG. 1 and/or camera 206 of FIG. 2 and may be generated and/or analyzed in accordance with executable instructions for calculating clotting time 110 of FIG. 1.

The electronic device 102 in accordance with executable instructions for calculating clotting time 110 may extract an initial frame. The initial frame may be a frame of a video captured by the camera 104. The electronic device 102 in accordance with executable instructions for calculating clotting time 110 may identify a cup holder (e.g., cup attachment portion 122) or cup (e.g., cup 116) in the initial frame image. The pixels corresponding to the cup 116 and/or cup attachment portion 122 may be isolated from other pixels within a given video frame Fₜ=[Rₜ, Gₜ, Bₜ]. In some examples, the cup 116 and/or cup attachment portion 122 may be marked with a marking that may facilitate identification of the corresponding pixels in one or more image(s) 112. For example, the cup 116 and/or cup attachment portion 122 may be colored a particular color (e.g., coated in blue ink). For example, the executable instructions for calculating clotting time 110 may specify color thresholding which may be used to identify the cup holder in a captured image. For example, an RGB color filter may be used to identify pixels corresponding to a color of the cup 116 and/or cup attachment portion 122 (e.g., blue). The output of this operation is pixels which correspond to the cup holder and/or cup. Additional spurious pixels may be included in the output that may share similar characteristics (e.g., same color range).

In some examples, spurious pixels may be removed and/or adjusted to provide an image of the cup holder. For example, the electronic device 102 may remove and/or adjust pixels not belonging to the cup holder in accordance with the executable instructions for calculating clotting time 110. To isolate the pixels corresponding to the cup attachment portion 122 and/or cup 116 from spurious ones, a flood-fill algorithm may be used to find a list of connected components in the frame. This algorithm defines a connected component as regions where all pixels in the region are connected along the horizontal, vertical and/or diagonal axis. The largest connected component is then selected as the most likely candidate region for the cup holder and/or cup. FIG. 3 shows these connected components, where, due to noise, this algorithm returned multiple connected components representing not just the cup holder but also spurious pixel regions. The list of connected components is then filtered to a single connected component representing the cup attachment portion 122 and/or cup 116.

In some examples, dimensions of the identified cup holder may be verified to ensure image processing has been accurate. To do this, the executable instructions for calculating clotting time 110 or other executable instructions may specify that a bounding box, bbox, around the candidate object is more than a particular expected size (e.g., 500×500 pixels in size in one example) to ensure the connected components filter has not returned any small pixel regions that correspond to spurious noise. Further, to ensure that the pixel region has a shape matching an expected shape of the cup attachment portion 122 and/or cup 116 (e.g., circular in some examples), the difference between the width and height of the bounding box may be checked to ensure it matches the expected shape (e.g., no greater than 200 pixels in some examples).

For example, known and/or expected dimensions of the cup attachment portion 122 may be stored (e.g., in memory 108 of FIG. 1). Dimensions calculated based on the image including the identified cup holder may be compared with the known dimensions. If the dimensions are within a tolerance threshold, the techniques may proceed. In some examples, if the dimensions are not within a tolerance threshold, and error may be displayed and/or the cup holder may again be attempted to be identified, in some examples utilizing a different initial frame of a captured video.

Examples of electronic devices described herein may identify a time tₛₜₐᵣₜ at which to start a clotting time measurement. The time may be identified in accordance with executable instructions for calculating clotting time 110 in some examples. In some examples, the start time may be specified by a user or other process (e.g., by providing a user input and/or receiving a start signal from another process). In some examples, the start time is identified by automatically detecting when a delivery device (e.g., capillary tube and/or pipette) has entered and moved out of the image(s) 112, indicating that the sample and/or activator substance (e.g., tissue factor) has been delivered to the cup 116. Accordingly, executable instructions for calculating clotting time 110 and/or other executable instructions may include executable instructions for identifying a start time.

In some examples, to capture the motion of the delivery device and separate it from the particle's motion, pixels within the target area defined by the bounding box described above (e.g., bbox) may be tracked, but not necessarily pixels within the cup containing the vibrating particle, as shown in FIG. 3. For example, the amount of information channels used for processing may be reduced. The electronic device 102 (e.g., processor 106 in accordance with the executable instructions for calculating clotting time 110) may convert multiple (e.g., each) RGB video frame with color channels R, G, B at timepoint t to a frame with grayscale intensity values. A mask, mask, may be identified that captures the interior of the cup and particle. The mask may generally be a shape that is a similar shape as an edge of the cup (e.g., circular in the example of FIG. 3). This mask is placed at the center of the bounding box bbox. The diameter of this mask may be a fraction (e.g., half) the bounding box's length or width, whichever is greater. Accordingly, the frame includes pixels that represent the cup and cup holder, and only the interior of the cup with the particle is masked out. Accordingly, only an edge of the cup 116 and/or cup attachment portion 122 is tracked to identify a presence of a delivery device. Accordingly, a mask may be created (e.g., by electronic device 102 in accordance with executable instructions for calculating clotting time 110) that may be centered at a bounding box. The bounding box may, for example, be defined based on the cup holder identification. The mask, which may be a same shape as the container or cup (e.g., circular), may be used to detect motion of a pipette or other tube into or proximate the cup. In this manner, systems described herein may detect the presence and/or motion of a pipette or tube (e.g., capillary tube) being brought proximate the cup to introduce sample and/or activator substance. In some examples, the presence of such a pipette or tube may provide an initial start time used to calculate clotting time or related parameters described herein.

The frame may be cropped to the location of bounding box bbox to provide a frame including only pixels representing an edge region of a cup and/or cup attachment portion. This may be referred to a tubeframe, M_{tube,t}, where M_{tube,t,i,j} represents the pixel at location (i, j).

$${M}_{t ube , t , i , j} = \{ \begin{matrix}0 & ( i , j ) ∈ mask \\ {F}_{t , i , j} & otherwise\end{matrix} \begin{matrix} \\ \end{matrix} \qquad Equation 1$$

Next, a motion of a delivery device (e.g., capillary tube or pipette) may be quantified between two masked frames (e.g., M_{tube,t}, M_{tube,t+τ}). Doing so may provide for a determination when the tube has entered the frame, which may be used to calculate tₛₜₐᵣₜ. The motion between two masked frames may be quantified by taking the L1-norm (e.g., the sum of the absolute difference between pixel intensities) between frames of dimension W x H, to generate a motion curve, d_{tube}[t]:

$${d}_{tube} [ t ] = {\sum }_{i = 0}^{W} {\sum }_{j = 0}^{H} \vert {M}_{t u b e , t , i , j} - {M}_{t u b e , t + τ , i , j} \vert , t ∈ [ 0 , 6 … , 60 ] \qquad Equation 2$$

The video is captured at 60 frames per second in some examples, and this motion value may be computed between frames at every τ interval every six frames, e.g., τ=6. This allow for capture of motion at a resolution of 100 ms. Other intervals and resolutions may be used in other examples. The motion curve is computed for a portion of the video (e.g., the first ten seconds of the video in some examples) within which the activator is expected to be added, such as an initial portion. tₛₜₐᵣₜ may then be calculated using this motion curve, d_{tube}[t]. For example, a moving average filter may be applied with a window size of 10 in some examples to d_{tube}[t]. The tube motion curve may be cropped of trailing motion artifacts that occur after the tube enters and exits the frame. This may be performed by finding the knee of the curve. Generally, the knee (or elbow) of a curve may refer to a point or region where the curve transitions from a high to a low slope region. In this case, the high slope region refers to the motion of the delivery device such as pipette or capillary tube, and the low slope one refers to the subsequent period of no motion or small motion artifacts, which may be cropped out.

The knee point of a motion curve d[t₀ . . . N] may be defined as the point p where the combined root mean square error (RMSE) of a linear regression on segments d[t_{0 . . . p-1}] and d[t_{p+1 . . . N}] is jointly minimized. Linear regression on d[t_{0 . . . p-1}] and d[t_{p+1 . . . N}] yield the coefficient vector (slope, intercept), \vec{β}_{0,p-1} and \vec{β}_{p+1,N}, respectively. The coefficients may be computed using least squares estimation:

$${\vec{β}}_{m , n} = {( {X}_{m , n}^{T} {X}_{m , n} )}^{- 1} {X}_{m , n}^{T} d [ {t}_{m … n} ] \qquad Equation 3$$

Here, Xm,n is a Vandermode matrix with the following entries:

$${X}_{m , n} = [ \begin{matrix}1 & m \\ 1 & m + 1 \\ ⋮ & ⋮ \\ 1 & n\end{matrix} ] \qquad Equation 4$$

The knee point may then be computed by minimizing the error function J(p, d[t]) below:

$${min}_{p} J ( p , d [ t ] ) = \sqrt{{Σ ( d [ {t}_{0 … p - 1} ] - {X}_{0 , p - 1} \cdot {\vec{β}}_{0 , p - 1} )}^{2}} + \sqrt{{Σ ( d [ {t}_{p + 1 … N} ] - {X}_{p + 1 , N} \cdot {\vec{β}}_{p + 1 , N} )}^{2}} \qquad Equation 5$$

Equation 5 may be minimized, for example, by iterating through all points, p, to obtain the knee point. Once the motion curve, d_{tube}[t], has been cropped to the knee point, the peak identifying the entry of the tube into the frame can be identified. The peak with maximum prominence in this cropped range represents the point of maximum motion change between frames and is marked as tₛₜₐᵣₜ.

The operations described herein with respect to locating tₛₜₐᵣₜ may be performed, for example, by electronic device 102 of FIG. 1. The executable instructions for calculating clotting time 110 may include executable instructions for calculating tₛₜₐᵣₜ as described, and may be executed by processor 106 to calculate tₛₜₐᵣₜ.

During operation, the particle may be identified in one or more video frames. 2D motion between video frames (e.g., consecutive video frames and/or between a video frame and a selected subsequent video frame) may be measured by systems described herein (e.g., using electronic device 102 in accordance with executable instructions for calculating clotting time 110). The motion may be measured, by, for example, measuring a number of pixels between a position of a center (or other portion) of a particle in one frame and the position of the center (or other portion) of the particle in a subsequent frame.

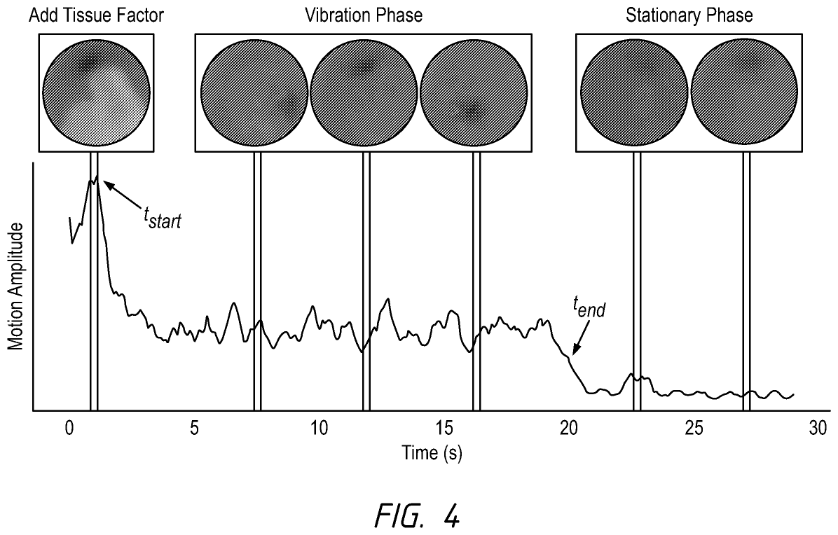

FIG. 4 is a plot of particle amplitude over time during operation of an example system arranged in accordance with examples described herein. The plot may represent, for example, motion of particle 124 of FIG. 1 and/or particle 214 of FIG. 2 responsive to vibration of the electronic device 102 of FIG. 1 and/or smartphone 204 of FIG. 2.

FIG. 4 illustrates a motion amplitude over time. The motion amplitude refers, for example, to a 2D distance between position of the particle between images (e.g., frames). The images may be consecutive and/or non-consecutive images. In some examples, images (e.g., frames) taken 100 ms apart are used, although other intervals may be used in other examples. The distance may be calculated and the clotting time determined using, e.g., electronic device 102 in accordance with executable instructions for calculating clotting time 110.

In the example of FIG. 4, the electronic device 102 may identify a start time tₛₜₐᵣₜ when a sample is added to the cup 116 and/or when an activator substance (e.g., a tissue factor) is added to the cup 116. As described with reference to FIG. 3, image processing techniques may be used to identify the presence of a pipette, tube, or other delivery device proximate the cup. The detection of the delivery device may indicate the start time tₛₜₐᵣₜ. Examples of techniques used to identify tₛₜₐᵣₜ are described herein.

During a next phase, when the sample is in a non-clotted state, an amplitude of the particle may remain relatively constant or somewhat variable until clotting increases viscosity of the fluid. At a time that the motion amplitude declines by greater than a threshold amount, the end time may be determined, tend. The end time may be determined using, for example electronic device 102 of FIG. 1. The executable instructions for calculating clotting time 110 may include executable instructions for identifying the end time, which may be executed by processor 106.

In some examples, the end time may be determined based on when the motion amplitude as declined to a particular percentage of the average motion amplitude during initial vibration (e.g., less than 50%, less than 40%, less than 30%, less then 25%, or other thresholds). In some examples, the end time may be determined based on when the motion amplitude has declined to a certain level (e.g., zero or stasis).

In some examples, tend may be defined as a point when the particle in the cup transitions from a moving state to a stationary state. To isolate the movements of vibration within the cup from the rest of the frame, the frame Ft may be cropped to within the bounding box around mask (e.g., as the bounding box and mask are described herein). The mask may then be applied so only pixels within the region (e.g., circular) of the cup are visible. This provides M_{particle,t}, where M_{particle,t,i,j} represents the pixel at location (i, j):

$${M}_{particle , t , i , j} = \{ \begin{matrix}0 & ( i , j ) ∉ mask \\ {F}_{t , i , j} & otherwise\end{matrix} \begin{matrix} \\ \end{matrix} \qquad Equation 6$$

To quantify the motion of the particle vibrating within the cup, a motion curve d_{particle[t]} may be calculated (e.g., by the electronic device 102) for the particle. This curve quantifies motion between a pair of masked frames (e.g., M_{particle,t}, M_{particle,t+τ}) for the full length of the video (t∈[0, N]), where N is the total number of frames in the video:

$${d}_{particle} [ t ] = {\sum }_{i = 0}^{W} {\sum }_{j = 0}^{H} \vert {M}_{p a r t i c l e , t , i , j} - {M}_{particle , t + τ , i , j} \vert , t ∈ [ 0 , N ] \qquad Equation 7$$

Next, tend, which may be the end time of the PT measurement, may be calculated from the motion curve d_{particle[t]}. The particle's motion curve may be smoothed with a moving average filter with window size (e.g., window size of ten in some examples). The process may only analyzes the particle's motion beginning a delay time (e.g., 10 seconds) after the start of measurement. The delay time (e.g., 10 s) may correspond to the lowest INR value of 0.8 that can be detected on the commercial POCT coagulometer used for testing, and it may represent a lower bound on PT values. A period of time after the particle has stopped moving may be removed from analysis. To do this, the particle motion curve may be normalized in the range of [0, 1]. The trailing portion of the curve may be cropped after the curve amplitude drops below the 0.01 mark. The knee point of the cropped motion curve of d_{particle[t]} corresponds to the point where the particle transitions from motion to a stationary state and is determined as tend, the end of the PT measurement.

A difference between the start time tₛₜₐᵣₜ and the end time tend may be referred to as a clotting time and may be used to calculate PT, INR, and/or other values relating to clotting behavior. The electronic device 102 of FIG. 1 may be used to calculate clotting time and/or any values relating to clotting behavior, such as PT, INR, and/or other values. The executable instructions for calculating clotting time 110 may include executable instructions for calculating clotting time, including PT, INR, and/or other values. The executable instructions for calculating clotting time 110 may be executed by processor 106.

The PT value may be computed by computing the time it takes for the particle to stop moving in the blood or plasma sample, PT=t_{end}−tₛₜₐᵣₜ. INR levels may be used since it normalizes the PT values across different labs and different test methods and may allow for easier comparison. INR is traditionally computed as

$$INR = {( \frac{P {T}_{t e s t}}{P {T}_{n o r m a l}} )}^{ISI + α} \qquad Equation 8$$

Here, PTₙₒᵣₘₐₗ is the PT value corresponding to plasma samples in the normal PR range; ISI is the international sensitivity index, which compares the batch of tissue factor used with an international reference tissue factor; and a is a correction factor. For plasma testing, a value of 16 s for PTₙₒᵣₘₐₗ was calculated in some examples as the average PT measurements from example systems, such as system 100 across 33 plasma samples within a normal INR range of 0.9-1.1. The ISI for an example lot of thromboplastin used was 1.23. A correction factor a of 0.48 was used to normalize the calculated INR values for an example system. For the coagulopathy evaluation, PTₙₒᵣₘₐₗ was set to 16 s similar to the first evaluation on plasma. The ISI for an example thromboplastin lot was 1.31, and a correction factor of 0.2 was used. For whole blood testing, PTₙₒᵣₘₐₗ was set to 12 s, which corresponds to an INR value of 1.0 on the commercial PT/INR test meter. The ISI for the thromboplastin lot used in some examples was 1.31, and a correction factor α of −0.31 was used. Other ISI and correction factors may be used in other examples.

The clotting time and/or PR/INR values or other values may be displayed, such as on a display of the electronic device 102 and/or smartphone 204. In some examples, the clotting time or other values may be stored (e.g., in memory 108 of FIG. 1) and/or may be transmitted (e.g., using electronic device 102 and/or smartphone 204) to another communication device or computing system.

In some examples, the electronic device 102 may provide an alert if the clotting time and/or PR/INR measurement is outside a tolerance range. For example, an audible, tactile, or visual alert may be provided. In some examples, a medication dosage (e.g., warfarin dosage) may need to be adjusted based on the clotting time measurement. For example, if the clotting time is greater than a desired time, an updated warfarin dosage (e.g., a decreased dosage) may be recommended. The recommended dosage may be displayed, stored, and/or transmitted to another computing system. If the PT/INR is less than a desired time, an updated warfarin dosage (e.g., a increased dosage) may be recommended. A patient may be alerted to contact their physician (or the physician may be automatically alerted) if the value is outside of the desired range as the patient is likely to be at higher risks of complications and undesirable outcomes when their values are outside the recommended range.

An advantage of leveraging smartphone hardware for medical purposes is that custom electronics and hardware may not have to be designed, which may lower the development costs typically required to obtain regulatory approval. Specifically, under the FDA's Mobile Medical Applications (MMA) and Software as a Medical Device (SaMD) guidance, the agency does not regulate the smartphone hardware, and only regulates custom software functions. The FDA has cleared or approved commercially available MMAs that use sensors like microphones and cameras to perform medical diagnostics. Accordingly, it may be advantageous to utilize commercially available smartphone hardware as described herein.

### Implemented Examples

An example implemented design was low-cost, with a total material cost of around $0.03, and used only a lightweight (17 g), compact (70×27.5×60.9 mm), 3D-printed plastic smartphone attachment, disposable plastic cup, and tiny copper particle. The components were arranged as described herein with reference, for example, to FIG. 1. The example system leveraged a smartphone's onboard vibration motor to vibrate a small silicone cup. The smartphone was coupled to the cup via a custom 3D-printed plastic attachment, as shown schematically in FIG. 1 and an image of the system is shown in FIG. 2.

The example system leverages the smartphone's onboard vibration motor to vibrate a small silicone cup. The smartphone is coupled to the cup via a custom 3D-printed plastic attachment. The ‘L’ shaped structure of the plastic attachment is constructed using thin material to allow the smartphone's vibrations to propagate to the cup holder while reducing dampening. In addition, the cup holder makes physical contact with both the bottom and sides of the cup to ensure maximal physical energy transfer from the attachment to the cup. The plastic attachment is designed to position the cup under the smartphone's camera and its width is determined by the smartphone's dimensions.

In the example of FIG. 2, the cup holds 10-20 μl of plasma or whole blood and a small copper particle. A thromboplastin activator is added to this mixture to activate the extrinsic pathways of the coagulation cascade. The copper particle is a 1 mm long AWG22 copper wire coated with dark blue ink for increased visibility. It is lightweight and moves freely in response to the phone's vibration. In addition, it is non-porous, which prevents it from soaking up any reagent. Copper material was used in this example because it is also low-cost, widely available, and easily cut to short lengths.

In the example of FIG. 2, the smartphone attachment for the Samsung Galaxy S9 included a 3D-printed plastic (PLA) frame that couples to the front of the smartphone, a 60 mm long vertical stem and a container holder. The length of the vertical stem was selected so that the container and particle were within the smartphone camera's focal length. The container holder has a diameter of 6 mm to accommodate the diameter of the container. The container was a silicone tube with an inner diameter of 4 mm, an outer diameter of 6 mm, and a height of 10 mm. Hot glue was used to create a water-tight seal around the end of the silicone tube. The Samsung Galaxy S8 has a similar geometry as the Galaxy S9, and the attachment used for the S9 fit on the S8.

When the blood or plasma sample is not coagulated, the smartphone's vibrations cause the copper particle to move and rotate within the sample. As the sample coagulates, its increasing viscosity constricts the particle's movements, slowing its projected 2D motion as seen by the camera. The particle's movement is recorded optically by the smartphone camera and analyzed to calculate PT/INR. The system identifies when the activator is added and computes the coagulation time automatically. The system isolates the 2D movement of the particle from the background and performs a correlation analysis of video frames to generate motion curves.

The start of a measurement is detected when the activator is dispensed to the cup. A steep drop in the particle's motion curve (such as shown in FIG. 4) indicates the particle's stationary state and marks the mixture's clot time. These motion curves are then analyzed to algorithmically identify the start and end time points of the measurement and compute the PT/INR values. The PT/INR result is displayed provided it passes checks on the data to ensure the test has been performed properly.

The example system processes video frames captured every 100 ms to calculate PT/INR. Accordingly, a smartphone camera that can capture video at 10 fps or greater is able to record the clotting process.

The system was analyze using 140 anonymized plasma samples from the University of Washington Medical Center (UWMC). A subset of these samples included plasma from patients who were undergoing treatment at the UWMC Anticoagulation clinic and were thus likely to have high PT/INR values. Samples were marked with PT/INR values obtained from a clinical-grade coagulation analyzer (Diagnostica Stago STA R Max). They were collected and tested on the smartphone setup within 12 h of being drawn from the patients. The plasma samples were stored at room temperature prior to measurement on the system. The PT/INR values of the plasma samples ranged from 11.4-49.8 s and 0.9-5.3, respectively, with a mean value of 20.1 s and 1.8 and a median value of 17 s and 1.4.

Since the smartphone measurements were performed hours after the plasma samples were collected, the samples (and the thromboplastin activator) were heated in a water bath at 37° C. (approximating the body temperature) for three minutes. The measurement was then conducted at room temperature and humidity. Twenty microliters of the plasma was added into the cup with the copper particle and placed into the smartphone attachment. The smartphone's vibration motor was turned on to vibrate continuously, and the camera began recording. Forty microliters of the activator was then added into the cup. Samples were tested on a Samsung Galaxy S9 phone and each test was performed twice to evaluate test-retest performance.

The PT/INR values computed by the smartphone system were compared against the laboratory PT/INR values. The inter-class correlation coefficient for PT/INR was R=0.963 and R=0.966, respectively. This is within the accuracy range of 0.77-0.97 for commercial point-of-care testing coagulometers. Bland-Altman analysis demonstrated a bias error of 1.617 s for PT, with 16 of 280 measurements samples falling outside the 95% agreement limits. Similar analysis for INR showed a bias error of −0.003, with 17 of 280 samples falling outside the 95% agreement limits. Increased variance was present at higher INRs may be due to variability in the international sensitivity index (ISI) of the tissue factor used by the laboratory versus the smartphone. To evaluate test-retest reliability, each plasma sample was tested twice. The intra-assay coefficient of variation (CV) between duplicate measurements of 140 plasma samples was 3.62% for PT and 6.14% for INR, which is within the range of 1.4-8.4% found using commercial point-of-care testing coagulometers.

For 100 of these plasma samples, a manual tilt-tube test was also conducted in parallel with the smartphone system. For these tests, 50 μl of plasma and 100 μl of activator were used; larger amount of plasma enabled more consistent testing with manual readings. The container was tilted back and forth until a clot formed. Clot times were noted by eye and recorded using a stop watch. Head-to-head testing demonstrated a PT/INR correlation between the manual test and the ground truth of R=0.960 for both PT/INR, which is similar to the correlation obtained by the smartphone system. The Bland-Altman analysis for PT showed a bias of −1.140 s, with five of 100 measurements falling outside the 95% agreement limits. Similar analysis for INR showed a bias of 0.098, with six of 100 measurements falling outside these limits.

The system was also evaluated on an additional 79 plasma samples collected from two sites, from patients with a known coagulopathy. Specifically, samples were obtained across a broad range of coagulopathic causes including patients who had disseminated intravascular coagulation (DIC) (n=8), liver disease (n=19), trauma (n=8), or conditions requiring anticoagulation such as extracorporeal membrane oxygenation (ECMO) (n=6) or who were on heparin (n=17) or warfarin (n=13) to treat a medical condition. The samples were obtained from both UWMC and Harborview Medical Center (HMC), and included samples from trauma patients in the emergency department undergoing blood transfusion. The PT/INR values of the plasma samples ranged from 12.6 to 67.2 s and 1-7.6, respectively, with a mean value of 22.2 s and 2.0 and a median value of 19.2 s and 1.6. The mean PT/INR for these patients is double that of a normal INR of 1.0. Samples were collected and tested using the same procedure as the first evaluation on plasma samples.

The inter-class correlation coefficient between the smartphone system and the clinical-grade coagulation analyzer was R=0.974 for both PT/INR. For samples with an elevated INR>1.2, the correlation coefficient within each of the coagulopathy categories ranged from 0.890 to 0.977. Bland-Altman analysis demonstrated a bias error of −1.865 s for PT, with six of 158 measurements samples falling outside the 95% agreement limits. Similar analysis for INR showed a bias error of 0.060, with nine of 158 samples falling outside the 95% agreement limits.

On subgroup analysis, patients who were on heparin therapy did not demonstrate a substantially elevated PT/INR with a mean of 13.9 s and 1.1 respectively. Heparin affects coagulation of the intrinsic pathway, while PT/INR assesses the extrinsic pathway so this is an expected negative control result. Warfarin does directly affect the extrinsic pathway and this group had the highest average PT/INR of the different conditions studied with a mean of 36.2 s and 3.6 respectively. Individuals with an INR>4.5 are at a nearly six fold increased risk of a bleeding event. Of the four patients with an INR>4.5, the system had an INR error of 14% compared to laboratory measurements. Patients were included who quickly developed coagulopathy after a traumatic event as well as those with longstanding liver disease or on anticoagulation therapy. In all tested cases, the PT/INR assessment tools were well correlated, indicating that this device can be used with a variety of coagulopathies.

The performance of the smartphone system was also evaluated on 80 anonymized samples of whole blood (blue top, 3 mL collected in sodium citrate) tested against the results of a commercial point-of-care PT/INR test meter (Coag-Sense, CoaguSense Inc.). In order to test the device capabilities across a range of coagulopathic conditions and PT/INR values, samples associated with particular diagnosis or from a particular clinic were preferentially obtained (the anticoagulation clinic and emergency department at Harborview Medical Center) with elevated PT/INR on laboratory testing, no other information about the sample than the patient coagulopathy was obtained. 30 of 80 samples were collected and tested on the smartphone system within 4 hours of being drawn from patients; twenty-two of the samples were tested within 4-12 h of blood draw; the remaining samples were refrigerated and tested more than 12 h later. The whole blood samples were collected under the same conditions as the plasma ones. PT/INR from the commercial test meter ranged from 13.5 to 39.0 s and 1.1-3.6, respectively, with a mean of 21.7 s and 1.9 and median of 21.1 s and 1.8.

The same amount of whole blood (10 μl) and thromboplastin activator (20 μl) were used for testing with the smartphone system and the commercial PT/INR meter. With the commercial meter, the whole blood and activator were each added to the test strip in quick succession as soon as the measurement started. The same thromboplastin activator was used for the commercial PT/INR meter and the smartphone system. As before, since each whole blood sample exceeded one milliliter, and each was tested twice with the smartphone to evaluate test-retest performance

The inter-class correlation coefficient was computed between the smartphone system and the commercial PT/INR meter. Across the 160 measurements of PT/INR, correlation coefficients were R=0.936 and R=0.933, respectively. These are within the accuracy range obtained by commercial point-of-care testing coagulometers. Bland-Altman analysis for PT showed a bias of −0.843 s, with nine of 160 measurements falling outside the 95% limits. The bias for INR was 0.007, with ten of 160 measurements falling outside these limits.

The test-retest performance was also evaluated for whole blood testing. The intra-assay CV between the duplicate measurements was 5.39% for both PT/INR, which again is within the precision range obtained from commercial point-of-care testing coagulometers.

The consistency of plasma and whole blood testing was evaluated in the system by measuring a low and high PT/INR sample ten times in a row. The low PT/INR (14.3 s and 1.1) plasma sample had a CV of 6.62% and 11.39%, respectively, while the high PT/INR (27.3 s and 2.5) sample had a CV of 4.52% and 7.76%, respectively. The low PT/INR (15.4 s and 1.3) whole blood sample had a CV of 8.06% for both PT/INR, while the high PT/INR (33.2 s and 3.0) sample had a CV of 6.64% for both PT/INR.

Accordingly, the smartphone-based micro-mechanical clot detection system demonstrated strong correlation with laboratory and point of care PT/INR tests for both plasma and whole blood. 279 of 280 (99.6%) plasma measurements and 100 of 100 whole blood measurements fall within the allowable differences for INR testing, greater than the 90% threshold set by the International Organization of Standardization for this type of device.

PT values were calculated as described herein based on tₛₜₐᵣₜ and tend time determinations described herein. For plasma testing, a value of 16 s for PTₙₒᵣₘₐₗ was calculated as the average PT measurements from the example system across the 33 plasma samples within the normal INR range of 0.9-1.1. The ISI for the lot of thromboplastin used was 1.23. A correction factor α of 0.48 was used to normalize the calculated INR values for the example system. For the coagulopathy evaluation, PTₙₒᵣₘₐₗ was set to 16 s similar to the first evaluation on plasma. The ISI for the thromboplastin lot was 1.31, and a correction factor of 0.2 was used. For whole blood testing, PTₙₒᵣₘₐₗ was set to 12 s, which corresponds to an INR value of 1.0 on the commercial PT/INR test meter. The ISI for the thromboplastin lot used in these experiments was 1.31, and a correction factor α of −0.31 was used.

A custom Android application was developed on a Samsung Galaxy S9 to perform measurements. The vibration motor on the Samsung Galaxy S9 has a resonant frequency of 159 Hz. The motor was set to vibrate continuously while the camera recorded the clotting process. The camera had an ISO of 320, 1/60 shutter speed, 5500 K white balance and captured frames at the maximum frame rate. For the benchmark experiments, an iOS app was also developed to vibrate the plastic attachment. The Samsung Galaxy S8, iPhone 5s and Google Pixel 3a phones used for benchmark experiments had a resonant frequency of 159, 231 and 153 Hz respectively. A vibration strength of 200 units was selected for the Samsung Galaxy S8 and S9 as this was high enough to move the particle freely without causing it to escape from the top of the container. The maximum vibration strength of 255 units was selected for the Google Pixel. The maximum vibration strength was also selected for the iPhone. The resonant frequency of the phone's vibration motor was fixed. The video recordings were captured at 30 fps with the default settings in the camera app of each phone. For benchmark experiments with different illuminance levels, ISO100 and a shutter speed of 1/1000 was used at 0 lux, while ISO800 and 1/30 shutter speed was used at other illuminance levels. 30 fps was used for testing different illuminance levels due to the high shutter speed selected.

From the foregoing it will be appreciated that, although specific embodiments have been described herein for purposes of illustration, various modifications may be made while remaining with the scope of the claimed technology.

Examples described herein may refer to various components as “coupled” or signals as being “provided to” or “received from” certain components. It is to be understood that in some examples the components are directly coupled one to another, while in other examples the components are coupled with intervening components disposed between them. Similarly, signal may be provided directly to and/or received directly from the recited components without intervening components, but also may be provided to and/or received from the certain components through intervening components.

What is claimed is:

1. A smartphone attachment comprising:
a cup;
an attachment member configured to contact a bottom and a side of the cup and a smartphone, wherein the attachment member is configured to transfer vibration from the smartphone to the cup, wherein the attachment member is further configured to position the cup within a field of view of a camera of the smartphone; and
a particle positioned in the cup and configured to move within the cup responsive to vibration from the smartphone.

2. The smartphone attachment of claim 1, wherein the particle comprises a copper wire.

3. The smartphone attachment of claim 1, wherein the particle comprises an iron particle.

4. The smartphone attachment of claim 1, wherein the cup is configured to receive a sample.

5. The smartphone attachment of claim 4, wherein a motion of the particle slows responsive to clotting of the sample.

6. A method comprising:
vibrate, using a motor of a smartphone, a container enclosing a sample and a measurement particle;
capture, using a camera of the smartphone, images of the measurement particle during vibration;
measure motion of the particle based on the images; and
calculate a clotting time based on the motion.

7. The method of claim 6, wherein the measurement particle comprises a copper particle or an iron particle.

8. The method of claim 6, wherein calculate the clotting time comprises determining a prothrombin time (PT) or international normalized ratio (INR).

9. The method of claim 6, wherein the sample comprises a whole blood sample or a plasma sample.

10. The method of claim 6, wherein the clotting time is calculated based on when the motion of the particle stops.

11. The method of claim 6, wherein the motion slows responsive to clotting in the sample.

12. The method of claim 6, wherein the motion is measured during a non-clotted state of the sample.

13. A system comprising:
a mobile terminal comprising a processor, a vibration motor, and a camera;
an attachment member configured to couple the mobile terminal to a cup; and
a particle positioned in the cup and configured to move within the cup responsive to vibration from the mobile terminal, wherein the vibration motor is configured to vibrate the attachment member, the camera is configured to capture images of a sample and the particle in the cup, and the processor is configured to calculate a clotting time of the sample in the cup based on the images.

14. The system of claim 13, wherein the mobile terminal comprises a smartphone.

15. The system of claim 13, wherein the attachment member is further configured to position the cup within a field of view of the camera.

16. The system of claim 13, wherein the particle comprises a copper particle.

17. The system of claim 13, wherein the particle comprises an iron particle.

18. The system of claim 13, wherein the processor is configured to calculate the clotting time based on a time when motion of the particle stops.

19. The system of claim 13, wherein the processor is configured to calculate the clotting time based on a slowing of a motion of the particle.

20. The system of claim 13, wherein the sample comprises a whole blood sample or a plasma sample.
